(19) United States
(12) Patent Application Publication
(10) Pub. No.: US 2001/0043914 A1
(43) Pub. Date: Nov. 22, 2001

(54) Methods and products for tumor immunotherapy

(76) Inventors: Edith Mathiowitz, Brookline, MA (US); Yong S. Jong, Warwick, RI (US); Jules S. Jacob, Taunton, MA (US); Nejat K. Egilmez, E. Amherst, NY (US); Richard B. Bankert, Eden, NY (US)

Correspondence Address: Helen C. Lockhart, Wolf, Greenfield & Sacks, P.C., 600 Atlantic Avenue, Boston, MA, 02210, US

(21) Appl. No.: 09/749,651

(22) Filed: Dec. 27, 2000

Related U.S. Application Data
(60) Provisional application No. 60/173,236, filed on Dec. 28, 1999.

(51) Int. Cl. A61K 38/20

(52) U.S. Cl. 424/85.2

(57) ABSTRACT

The invention relates to methods and products for preventing and treating tumors. In particular the invention relates to the use of slow release microparticles containing IL-12, which are directly injected into a tumor, in order to treat the tumor or to prevent tumor growth or metastasis.

### RELATED APPLICATION INFORMATION

This application claims the benefit of priority under 35 USC 119 to U.S. Provisional Patent Application No. 60/173,236 filed on Dec. 28, 1999 and entitled IN SITU TUMOR VACCINATION WITH INTERLEUKIN-12 ENCAPSULATED BIODEGRADABLE MICROSPHERES: INDUCTION OF TUMOR REGRESSION AND POTENT ANTITUMOR IMMUNITY. The entire contents of the provisional patent application are hereby incorporated by reference.

### FIELD OF THE INVENTION

The invention relates to methods and products for preventing and treating tumors. In particular the invention relates to the prevention and treatment of tumors by local administration of slow release microparticles containing IL-12.

### BACKGROUND OF THE INVENTION

The ability of cytokines to treat tumors has been proposed. Systemic bolus cytokine therapy, however, has been associated with low efficacy and severe side effects in the clinic (Ben-Efraim, S. Tumor Biology 20:1-24 (1999).). Delivery of genes encoding cytokines has been proposed in order to reduce some of the toxic effects of the cytokines. The attraction of gene-modification lies mainly in the fact that the cytokine of choice can be delivered to the tumor microenvironment in a paracrine manner, circumventing the severe side effects associated with systemic cytokine immunotherapy (Dranoff, G. J. Clin. Oncol. 16:2548-2556, 1998, Tuting, T., et al, J. Mol. Med. 75:478-491, 1997, Colombo, M. P. and Forni, G. Cancer and Met. Rev. 16:421-432, 1997). While some encouraging results have been reported with cytokine gene-modified tumor cell vaccines (Soiffer, R., et al. Proc. Nat. Acad. Sci. 95:13141-13146, 1998), it has also become increasingly clear that with the possible exception of melanomas, the current gene transfer technologies lack the simplicity and the versatility required for universal clinical application (Dranoff, G. J. Clin. Oncol. 16:2548-2556, 1998, Colombo, M. P. and Forni, G. Cancer and Met. Rev. 16:421-432, 1997). The development of clinically more feasible and less expensive alternative technologies for the local and sustained delivery of cytokines to tumors can significantly enhance the clinical implementation of cytokine-based cancer immunotherapies.

### SUMMARY OF THE INVENTION

The invention relates in some aspects to improved methods for treating and preventing tumors by administering IL-12 directly to the tumor in a microparticle. It is believed that the sustained release of IL-12 to the tumor microenvironment will induce the development of an antitumor inflammatory reaction followed by massive tumor cell death, release of tumor antigens and the development of a systemic long-term antitumor immunity. Thus in one aspect the invention is a method for in situ tumor vaccination of a subject. The method involves administering to a tumor of a subject an effective amount for preventing tumor growth of a microparticle preparation containing IL-12, wherein an antigen is not co-administered to the subject. In one embodiment between 1 and 100% of the pro-inflammatory cytokine is released from the microparticle and preferably it is all bioactive.

In another aspect, the invention is a method for in situ tumor vaccination of a subject. The method involves administering to a site of a tumor of a subject an effective amount for preventing tumor growth of a microparticle preparation containing IL-12, the microparticles of the microparticle preparation have an average particle size of between 10 nanometers and 10 microns.

According to another aspect, the invention is a method for in situ tumor vaccination of a subject by administering to a site of a tumor of a subject an effective amount for preventing tumor growth of a microparticle preparation containing IL-12, the microparticle of the microparticle preparation having been prepared by phase inversion nanoencapsulation.

In yet another aspect, the invention is a method for in situ tumor vaccination of a subject, by administering to a site of a tumor of a subject an effective amount for preventing tumor growth of a microparticle preparation containing IL-12, wherein the microparticle preparation is administered to the subject during or following a medical procedure to remove or kill the tumor cells. Optionally the medical procedure is a surgical procedure, a chemotherapeutic procedure, or an immunotherapeutic procedure.

A method for preventing tumor metastasis in a subject is provided according to other aspects of the invention. The method involves administering to a site of a tumor of a subject in need thereof an effective amount for preventing tumor metastasis of a microparticle preparation containing IL-12.

In another aspect, the invention is a method for effecting tumor regression in a subject, by administering to a site of an established tumor of a subject in need thereof an effective amount for effecting tumor regression of a microparticle preparation containing IL-12.

A method for in situ tumor vaccination of a subject by administering to a tumor of a subject a microparticle preparation containing an effective amount of IL-12 and a cytokine that augments antigen processing and presentation is provided according to another aspect of the invention. Preferably the effective amount of IL-12 and the cytokine that augments antigen processing and presentation results in a synergistic prevention of tumor cell growth. In one embodiment the IL-12 and the cytokine that augments antigen processing and presentation results in a synergistic prevention of metastasis. In another embodiment the cytokine that augments antigen processing and presentation is GM-CSF.

In yet other aspects, the invention relates to a method for in situ tumor vaccination of a subject. The method involves administering to a site of a tumor of a subject an effective amount for preventing tumor growth of a microparticle preparation containing IL-12, wherein between about 0.1% and 20% of the IL-12 released from the microparticle preparation in vivo is bioactive. Preferably between about 5% and 10% of the IL-12 released from the microparticle preparation in vivo is bioactive. In other embodiments about 8% of the IL-12 released from the microparticle preparation in vivo is bioactive.

According to another aspect, a method for in situ tumor vaccination of a subject, by administering to a site of a tumor of a subject an effective amount for preventing tumor growth of a microparticle preparation containing IL-12, wherein the microparticle preparation has an IL-12 release rate of between about 60 pg/μg of particle/day and 3400 pg/μg of particle/day is provided. In one embodiment the microparticle preparation has an IL-12 release rate of between about 250 pg/μg of particle/day and 1000 pg/μg of particle/day. In other embodiments the microparticle preparation has an IL-12 release rate of about 550 pg/μg of particle/day. Optionally the IL-12 is released from the microparticle preparation over a period of between about 3 days and 2 months, between about 8 days and 1 month or between about 12 days and 15 days.

In some embodiments, the microparticle preparation is administered to the subject prior to a medical procedure to remove or kill the tumor cells. In other embodiments the microparticle preparation is administered to the subject during or following a medical procedure to remove or kill the tumor cells. The medical procedure may be, for instance, a surgical procedure, a chemotherapeutic procedure, or an immunotherapeutic procedure.

In other embodiments, the method also involves administering to the subject a tumor antigen. The tumor antigen may be, for instance, a tumor cell suspension, a purified antigen, or a recombinant antigen.

The invention also involves in some embodiments the use of microparticles in which between about 0.1% and 20% of the IL-12 released from the microparticle preparation in vivo is bioactive. Optionally, between about 5% and 10% or about 8% of the IL-12 released from the microparticle preparation in vivo is bioactive.

In other embodiments the microparticle preparation has an IL-12 release rate of between about 60 pg/μg of particle/day and 3400 pg/μg of particle/day, between about 250 pg/μg of particle/day and 1000 pg/μg of particle/day or of about 550 pg/μg of particle/day. In other embodiments the IL-12 is released from the microparticle preparation over a period of between about 3 days and 2 months, between about 8 days and 1 month, or between about 12 days and 15 days.

In some embodiments the microparticles of the microparticle preparation have an average particle size of between 10 nanometers and 10 microns. The microparticle of the microparticle preparation is prepared by phase inversion nanoencapsulation in yet other embodiments.

In some other aspects the methods of the invention involve the administration of the cytokine directly to lung tissue.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a set of bar graphs depicting the effects of in vitro release of cytokines (recombinant human PEG-IL-2 (1B), murine IL-12 (1A) and murine GM-CSF (1C)) from microspheres. The bioactivity of IL-12 that was released from the microspheres was determined to be 2.2×10⁵ units/mg using a murine splenocyte proliferation assay. Each data point was measured in triplicate. Bars=standard deviation.

FIG. 2 is two graphs (2A is % tumor free mice and 2B is tumor volume) demonstrating the effect of IL-12 microspheres on Line-1 tumor engraftment and growth of established tumors in BALB/c mice. (A) Line-1 tumor cells (1×10⁶) and microspheres (50 μg) were mixed and injected subcutaneously in 100 μl of DMEM into BALB/c mice. Mice were scored as tumor positive when the diameter of the tumor was >3 mm (n=5). (B) Mice were injected with 1×10⁶ Line-1 cells subcutaneously and tumors were allowed to grow to ˜4 mm in diameter. Tumors were then injected directly with 2 mg of microspheres in 50 μl of DMEM using a 28.5 gauge needle and tumor growth was monitored weekly. Tumor volume was calculated based on the formula a²×b/2 where a and b are the shortest and the longest dimensions of the tumor, respectively (n=10 for BSA, PEG-IL-2 and IL-12 groups, and 5 for the GM-CSF group). The differences between the BSA, PEG-IL-2 and GM-CSF-treated groups were not significant at any time point (p>0.22) whereas the differences between the IL-12 group and the other groups were significant at weeks 3, 4 and beyond (p<0.002) in panel B. Bars=standard deviation.

FIG. 3 is a graph demonstrating the effect of IL-12 microspheres on the growth of established Line-1 tumors in CB.17 SCID mice. Established Line-1 tumors (˜4 mm in diameter) in CB.17 SCID or BALB/c mice were injected either with BSA or IL-12-loaded microspheres (2 mg/tumor in 50 μl DMEM) and tumor growth was monitored weekly (n=5). The differences between the IL-12 treated SCID mice and the IL-12+TMβ1-treated SCID mice are highly significant at weeks 2 and 3 (p <0.007). Bars=standard deviation.

FIG. 4 is two graphs (4A is a line graph and 4B is a bar graph) demonstrating the effect of IL-12-loaded microspheres on the growth of spontaneous lung metastases in BALB/c mice. BALB/c mice were injected with 5×10⁷ Line-1 cells in 200 μl DMEM subcutaneously in the ventral caudal midline on day 0. Tumors were allowed to reach a diameter of 7-8 mm and were treated with a single intratumoral injection of either BSA or IL-12-loaded microspheres (1 mg/mouse in 100 μl). (A) Growth of established tumors was monitored every 3 days. The differences between the BSA-treated and the IL-12-treated mice were significant on days 15 and 20 (p=0.04 and 0.001, respectively) (B) Mice were sacrificed 14 days after treatment and the lungs were examined for tumor nodules under a dissecting microscope (n=5). The differences between the no-treatment/BSA-treated groups and the IL-12-treated groups were highly significant (p=0.0036 and 0.0015, respectively). Bars=standard deviation.

FIG. 5 is two graphs (5A is a line graph and 5B is a bar graph) depicting recurrence and metastasis following preoperative vaccination of the primary tumor with IL-12 microspheres versus control. Bars=standard deviation, n=5.

FIG. 6 is two graphs depicting effect of preoperative vaccination with IL-12 and GM-CSF microspheres on the development of lung metastasis (6A) and tumor nodules (6B). Bars=standard deviation, n=5.

FIG. 7 is two graphs (7A is metastasis and 7B is # of lung nodules) demonstrating that vaccination with IL-12+GM-CSFd microspheres is superior to soluble 12+GM in the surgical metastasis model. Four of 6, 4 of 7 and 1 of 7 mice developed lung metastasis in the surgery only, bolus cytokine and microsphere groups, respectively.

### DETAILED DESCRIPTION

The invention is based in part on several surprising discoveries related to the direct injection of cytokines formulated in microspheres into tumors. It has been discovered that low doses of IL-12 released locally from the microspheres at a single tumor site in a sustained fashion have a significant antitumor effect resulting in the disappearance of the primary tumor, reduction in distant metastases and the development of systemic antitumor immunity.

Prior studies with IL-12 have established that the potent antitumor effects of IL-12 are tempered by the dose- and schedule-dependent toxicity in mice (Coughlin, et al Cancer Res. 57:2460-2467, 1997) and in humans (Leonard, J. P., et al Blood 90:2541-2548, 1997; Atkins, M. B., et al. Clin. Can. Res. 3:409-417, 1997) when administered systemically. The severe toxicity associated with systemic infusion of IL-12 in early clinical trials was partially alleviated by altering the schedule and dose of treatment but the lack of significant antitumor efficacy in these trials has been disappointing (Atkins, M. B., et al. Clin. Can. Res. 3:409-417, 1997, Robertson, M. J., et al. Clin. Can. Res. 5:9-16, 1999). Recent studies demonstrated that systemic administration of IL-12 also induces a transient generalized immunosuppression in mice (Kurzawa, H., et al Cancer Res. 58:491-499, 1998; Kurzawa Koblish, H et al J. Exp. Med. 188:1603-1610, 1998). Ineffectiveness of systemic IL-12 therapy in the clinic could be due to the inability of the cytokine to reach effective local concentrations in the tumor bed at maximum tolerated dose and/or the induction of a generalized suppression of T-cell responses. Moreover, relatively large quantities of IL-12 (in the range of 1-10 μg/day) are typically required. Therefore, administration of recombinant IL-12 at these dosages often resulted in toxicity.

As disclosed herein, we found that intratumoral injection of IL-12-loaded PIN/PLA microspheres, but not IL-2 or GM-CSF-loaded microspheres, induced the regression of established tumors, prevented spontaneous metastasis and promoted the development of tumor-specific immunity. Our observation that tumor cell engraftment can be reduced by injection of IL-2 in mice was consistent with other reports. The results observed with direct injection of IL-12, however, were surprising e.g., because other cytokines such as IL-2 which have known anti-tumor properties were dramatically less effective.

The work presented here establishes that biodegradable polymer microspheres can effectively deliver biologically active IL-12 to established tumors and thereby provoke a strong and lasting systemic antitumor immunity in several different embodiments of a weakly immunogenic syngeneic murine tumor model, whereas other cytokines, such as, IL-2 and GM-CSF were ineffective. This is the first report where complete tumor regression, suppression of spontaneous metastasis and the development of protective tumor-specific immunity is achieved using IL-12-loaded biodegradable microspheres, demonstrating the clinical effectivity of this technology. The effectivity of this approach was also confirmed in a syngeneic tumor model where complete regression of established Colon 26 tumors was achieved in 4 of 5 BALB/c mice following a single intratumoral injection of IL-12 microspheres. No tumor suppression was observed with control BSA-loaded microspheres in these experiments.

Our data further establish that vaccination of tumor-bearing mice with IL-12-loaded microspheres in situ is superior to vaccination of mice with mixtures of live tumor cells and IL-12 microspheres which in turn is more effective than irradiated tumor cell/microsphere mixtures in inducing protective antitumor immunity. This finding has important therapeutic relevance with respect to the design of vaccination strategies for cancer patients. Others have shown that vaccination with live cytokine gene-modified tumor cells is more effective than vaccination with irradiated cytokine gene-modified tumor cells and antigen dose has been suggested as a critical factor to explain these observations (Colombo, M. P. and Forni, G. Cancer and Met. Rev. 16:421-432, 1997; Cavallo, et al. J. Natl. Cancer Inst. 89:1049-1058, 1997). Surprisingly, in contrast to the earlier studies, it has been discovered that IL-12 loaded microspheres are more effective when administered alone in situ than when administered in combination with an exogenous antigen.

Thus, in one aspect the invention is a method for in situ tumor vaccination of a subject. The method is performed by administering to a tumor of a subject an effective amount for preventing tumor growth of a microparticle preparation containing IL-12. In this aspect the IL-12 microparticles are not co-administered to the subject with an antigen. Thus, the tumor vaccination may be accomplished in the absence of antigen.

IL-12 (interleukin-12), is a heterodimeric cytokine predominantly produced by macrophage, B lymphocytes, monocytes and dendritic cells and encoded by two separate genes, p40 and p35. IL-12 has been reported to enhance NK cell and CTL activity, to stimulate the differentiation of Th1 cells, and to induce production of cytokines, such as IFN-γ. (Gately, M. K., et al, Cell. Immunol. 143:127 (1992); Naume, B., et al, J. Immunol. 148:2429 (1992); Hsieh, C. S., et al Science 260:547 (1993); Manetti, R., et al J. Exp. Med. 177:1199 (1993); Chan, S. H., et al, J. Exp. Med. 173:869 (1991); D'Andrea, A., et al, J. Exp. Med. 1,76:1387 (1992); Macatonia, S. E., et al, Int. Immunol. 5:1119 (1993); Tripp, C. S., et al, Proc. Natl. Acad. Sci. USA 90:3725 (1993)). It has also been demonstrated to modulate T cell response to antigens (see, for example, PCT publication nos. WO 92/05256 and WO 90/05147).

As used herein the term “IL-12” refers to a peptide unless otherwise indicated. For instance, the term IL-12 only refers to a nucleic acid encoding IL-12 when used in the context of an IL-12 nucleic acid. IL-12 refers to intact IL-12, its individual subunits, fragments thereof which exhibit IL-12 activity and functional equivalents of IL-12. Functional equivalents of IL-12 include modified forms of IL-12 protein having similar activity to intact IL-12. The purification and cloning of IL-12 are disclosed in several references including PCT publication nos. WO 92/05256 and WO 90/05147, and in European patent publication no. 322,827.

IL-12 useful according to the invention can be obtained from any known source. For example, IL-12 can be purified from natural sources (e.g., human, animal), produced by chemical synthesis or produced by recombinant DNA techniques e.g., from nucleic acid sequences encoding IL-12.

IL-12 can be produced recombinantly through expression of DNA sequences encoding one or both of the IL-12 subunits in a suitable transformed host cell. In vitro synthesized coding sequences encoding IL-12 p35 and p40 subunits can readily be prepared in quantities sufficient for molecular cloning using standard recombinant molecular biological techniques, including PCR amplification and hybridization, using the published DNA sequences. For example, IL-12 nucleic acids are well known. p35-encoding DNA and peptide sequences have been deposited at GenBank under Accession numbers U19842, U19835, D63334, X97018, U83185, AJ271034, AF173557, and AJ271034. p40-encoding DNA and peptide sequences have been deposited at GenBank under Accession numbers AF004024, AF209435, S79628, AF007576, U83184, X97019, D63333, and U199834. Also, DNA sequences encoding human IL-12 are set forth in PCT/US91/06332.

Using known methods the IL-12 encoding DNA may be linked to an expression vector. Any suitable expression vector may be employed to produce IL-12 recombinantly. For mammalian expression, numerous expression vectors are known. Viral vectors are a preferred type of vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other vectors not named but known in the art. Descriptions of expression vectors are generally provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991). Specific examples of vecotrs include but are not limited to pED (Kaufman et al., Nucleic Acids Res. 19, 4484-4490(1991)), pEF-BOS (Mizushima et al., Nucleic Acids Res. 18, 5322 (1990)); pXM, pJL3 and pJL4 (Gough et al., EMBO J. 4, 645-653 (1985)); and pMT2 (derived from pMT2-VWF, A.T.C.C. #67122; see PCT/US87/00033). Suitable expression vectors for use in yeast, insect, and bacterial cells are also known. Construction and use of such expression vectors is within the ordinary level of skill in the art.

The expression vector containing the IL-12 subunits may then be transformed into a host cell, and protein expression may be induced to produce heterodimeric human IL-12. Suitable host cells for recombinant production of IL-12 include, for example, mammalian cells such as Chinese hamster ovary (CHO) cells, monkey COS cells, mouse 3T3 cells, mouse L cells, myeloma cells such as NSO (Galfre and Milstein, Methods in Enzymology 73, 3-46 (1981)), baby hamster kidney cells, and the like. IL-12 may also be produced by transformation of yeast, insect, and bacterial cells with DNA sequences encoding the IL-12 subunits, induction and amplification of protein expression, using known methods.

Alternatively, the IL-12 subunit-encoding sequences can be obtained from natural sources that produce IL-12. The IL-12 subunit-encoding sequences may be obtained or derived from other species which demonstrate sufficient sequence identity to be functionally equivalent to human IL-12, when the methods of the invention are being used to treat humans. For example, IL-12 is known to be produced by mice.

In order to provide IL-12 for therapeutic purposes it is preferred that the material be isolated. An isolated molecule is a molecule that is substantially pure and is free of other substances with which it is ordinarily found in nature or in vivo systems to an extent practical and appropriate for its intended use. In particular, the molecules, e.g., IL-12 or antigen are sufficiently pure and are sufficiently free from other biological constituents of host cells so as to be useful in, for example, producing pharmaceutical preparations. Because an isolated molecule of the invention may be admixed with a pharmaceutically-acceptable carrier in a pharmaceutical preparation, the molecule may comprise only a small percentage by weight of the preparation. The molecule is nonetheless substantially pure in that it has been substantially separated from the substances with which it may be associated in living systems. Methods for isolating and purifying IL-12 have been described, e.g., in U.S. Pat. No. 5,853,714.

The IL-12 is administered to a subject for treating or preventing cancer in the subject. A “subject” shall mean a human or vertebrate mammal including but not limited to a dog, cat, horse, cow, pig, sheep, goat, or primate, e.g., monkey.

The terms “cancer” and “tumor” are used interchangeably herein and refer to an uncontrolled growth of cells which interferes with the normal functioning of the bodily organs and systems. Cancers which migrate from their original location and seed vital organs can eventually lead to the death of the subject through the functional deterioration of the affected organs. Hematopoietic cancers, such as leukemia, are able to outcompete the normal hemopoietic compartments in a subject, thereby leading to hemopoietic failure (in the form of anemia, thrombocytopenia and neutropenia) ultimately causing death. Cancers and tumors include solid tumors, metastatic tumor cells, nonsolid cancers of the blood, marrow, and lymphatic systems, carcinomas (cancers derived from epithelial cells), sarcomas (derived from mesenchymal tissues) lymphomas (solid tumors of lymphoid tissues), and leukemias (marrow or blood borne tumors of lymphocytes or other hematopoietic cells).

Non-limiting examples of cancers are basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

A “subject having cancer” is a subject that has been diagnosed with a cancer. In some embodiments, the subject has a cancer type characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

The IL-12 is delivered to the site of a tumor in a microparticle preparation. Any type of microparticle known in the art may be used in the methods of the invention. The terms “microparticle”, “microsphere”, “nanoparticle” and “nanosphere” are used interchangeably to refer to polymeric particles having a size range of nanometers-micrometers. These materials are capable of biodegrading in the body. The microparticles may contain consistent formulations of polymer and cytokine or outer layers of polymer and inner core of cytokine or mixtures thereof. Polymers useful for preparing the microparticles of the invention include, but are not limited to, nonbioerodable and bioerodable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

One type of polymer is a bioadhesive polymer. A bioadhesive polymer is one that binds to mucosal epithelium under normal physiological conditions. Thus, bioadhesive polymers are useful for delivery of a substance to the mucosal epithelium. Although these polymers may be used to generate microparticles for delivery of the IL-12 directly into the tumor site, they are not necessary. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups primarily responsible for forming hydrogen bonds are the hydroxyl and the carboxylic groups. Numerous bioadhesive polymers are discussed in that application.

Representative bioadhesive polymers of particular interest include bioerodible hydrogels described by H. S. Sawhney, C. P. Pathak and J. A. Hubell in Macromolecules, 1993, 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly butylmethacrylate), poly(isobutylmethacrylate), poly(hexlmethacrylate), poly(isodecl methacrylate), poly(lauryl methacrylate), poly(phenyl methacrylate), poly (methyl acrylate), poly(isopropyl acrylate), poly(isobutyl acrylate), and poly(octadecl acrylate). Most preferred is poly(fumaric-co-sebacic)acid.

Polymers with enhanced bioadhesive properties can be provided wherein anhydride monomers or oligomers are incorporated into the polymer. The oligomer excipients can be blended or incorporated into a wide range of hydrophilic and hydrophobic polymers including proteins, polysaccharides and synthetic biocompatible polymers. Anhydride oligomers may be combined with metal oxide particles to improve bioadhesion even more than with the organic additives alone. Organic dyes because of their electronic charge and hydrophobicity/hydrophilicity can either increase or decrease the bioadhesive properties of polymers when incorporated into the polymers. The incorporation of oligomer compounds into a wide range of different polymers which are not normally bioadhesive dramatically increases their adherence to tissue surfaces such as mucosal membranes.

As used herein, the term “anhydride oligomer” refers to a diacid or polydiacids linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. In one embodiment, the diacids are those normally found in the Krebs glycolysis cycle. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example from toluene or other organic solvents. The oligomer is collected by filtration, and washed, for example, in ethers. The reaction produces anhydride oligomers of mono and poly acids with terminal carboxylic acid groups linked to each other by anhydride linkages.

The anhydride oligomer is hydrolytically labile. As analyzed by gel permeation chromatography, the molecular weight may be, for example, on the order of 200-400 for fumaric acid oligomer (FAPP) and 2000-4000 for sebacic acid oligomer (SAPP). The anhydride bonds can be detected by Fourier transform infrared spectroscopy by the characteristic double peak at 1750 cm⁻¹ and 1820 cm⁻¹, with a corresponding disappearance of the carboxylic acid peak normally at 1700 cm⁻¹.

In one embodiment, the oligomers may be made from diacids described for example in U.S. Pat. No. 4,757,128 to Domb et al., U.S. Pat. No. 4,997,904 to Domb, and U.S. Pat. No. 5,175,235 to Domb et al., the disclosures of which are incorporated herein by reference. For example, monomers such as sebacic acid, bis(p-carboxy-phenoxy)propane, isophathalic acid, fumaric acid, maleic acid, adipic acid or dodecanedioic acid may be used. Organic dyes, because of their electronic charge and hydrophilicity/hydrophobicity, may alter the bioadhesive properties of a variety of polymers when incorporated into the polymer matrix or bound to the surface of the polymer. A partial listing of dyes that affect bioadhesive properties include, but are not limited to: acid fuchsin, alcian blue, alizarin red s, auramine o, azure a and b, Bismarck brown y, brilliant cresyl blue ald, brilliant green, carmine, cibacron blue 3 GA, congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2, 3, 13 and 86, rose bengal, safranin o, Sudan III and IV, Sudan black B and toluidine blue.

The working molecular weight range for the polymer is on the order of 1 kDa-150,000 kDa, although the optimal range is 2 kDa-50 kDa. The working range of polymer concentration is 0.01-50% (weight/volume), depending primarily upon the molecular weight of the polymer and the resulting viscosity of the polymer solution. In general, the low molecular weight polymers permit usage of a higher concentration of polymer. The preferred concentration range will be on the order of 0.1%-10% (weight/volume), while the optimal polymer concentration typically will be below 5%. It has been found that polymer concentrations on the order of 1-5% are particularly useful.

The viscosity of the polymer solution preferably is less than 3.5 centipoise and more preferably less than 2 centipoise, although higher viscosities such as 4 or even 6 centipoise are possible depending upon adjustment of other parameters such as molecular weight. It will be appreciated by those of ordinary skill in the art that polymer concentration, polymer molecular weight and viscosity are interrelated, and that varying one will likely affect the others.

Recently, we have developed a technology referred to as phase inversion nanoencapsulation, (PIN) for highly efficient encapsulation of biologically active molecules into polymer microspheres (Mathiowitz, E., et al., Nature 386:410-414, 1997, U.S. Pat. No. 6,143,211). This spontaneous process does not require vigorous stirring/sonication during the formation of emulsions and labile proteins are efficiently encapsulated without denaturation or losses to aqueous non-solvent baths. We have demonstrated that recombinant human IL-2-loaded poly-lactic acid (PLA) microspheres prepared by PIN release physiologically relevant quantities of bioactive IL-2 for extended periods and that the in vivo release of IL-2 from the PLA microspheres provokes a mouse natural killer (NK) cell mediated suppression of human tumor xenografts in SCID mice (Egilmez, N. K., et al. Cancer Immunol. Immunother. 46:21-24, 1998).

Surprisingly good therapeutic effects have been observed when microparticles prepared by the PIN method or having similar properties are used according to the invention. Thus, it is preferred that microparticles of the invention are those which are prepared by PIN or have the properties of microparticles prepared by PIN.

The “phase inversion” of polymer solutions under certain conditions can bring about the spontaneous formation of discreet microparticles, including nanospheres. By using relatively low viscosities and/or relatively low polymer concentrations, by using solvent and nonsolvent pairs that are miscible and by using greater than ten fold excess of nonsolvent, a continuous phase of nonsolvent with dissolved polymer can be rapidly introduced into the nonsolvent, thereby causing a phase inversion and the spontaneous formation of discreet microparticles. The process can be performed very rapidly, the entire process taking less than five minutes in some cases. The actual phase inversion and encapsulation can take place in less than 30 seconds.

In the preferred processing method, a mixture is formed of the IL-12 to be encapsulated, a polymer and a solvent for the polymer. The cytokine to be encapsulated may be in liquid or solid form. It may be dissolved in the solvent or dispersed in the solvent. The cytokine thus may be contained in microdroplets dispersed in the solvent or may be dispersed as solid microparticles in the solvent. The loading range for the cytokine within the microparticles is between 0.01-80% (cytokine weight/polymer weight). When working with nanospheres, an optimal range is 0.1-5% (weight/weight).

The cytokine is added to the polymer solvent, preferably after the polymer is dissolved in the solvent. The solvent is any suitable solvent for dissolving the polymer. Typically the solvent will be a common organic solvent such as a halogenated aliphatic hydrocarbon such as methylene chloride, chloroform and the like; an alcohol; an aromatic hydrocarbon such as toluene; a halogenated aromatic hydrocarbon; an ether such as methyl t-butyl; a cyclic ether such as tetrahydrofuran; ethyl acetate; diethylcarbonate; acetone; or cyclohexane. The solvents may be used alone or in combination. The solvent chosen must be capable of dissolving the polymer, and it is desirable that the solvent be inert with respect to the cytokine being encapsulated and with respect to the polymer. The polymer may be any suitable microencapsulation material such as those described above.

The nonsolvent, or extraction medium, is selected based upon its miscibility in the solvent. Thus, the solvent and nonsolvent are thought of as “pairs”. We have determined that the solubility parameter (δ(cal/cm³)^{½}) is a useful indicator of the suitability of the solvent/nonsolvent pairs. The solubility parameter is an effective protector of the miscibility of two solvents and, generally, higher values indicate a more hydrophilic liquid while lower values represent a more hydrophobic liquid (e.g., δᵢwater=23.4 (cal/cm³)^{½} whereas δᵢhexane=7.3 (cal/cm³)^{½}). We have determined that solvent/nonsolvent pairs are useful where 0<δ solvent−δ nonsolvent<6(cal/cm³)^{½}. Although not wishing to be bound by any theory, an interpretation of this finding is that miscibility of the solvent and the nonsolvent is important for formation of precipitation nuclei which ultimately serve as foci for particle growth. If the polymer solution is totally immiscibile in the nonsolvent, then solvent extraction does not occur and nanoparticles are not formed. An intermediate case would involve a solvent/nonsolvent pair with slight miscibility, in which the rate of solvent removal would not be quick enough to form discreet microparticles, resulting in aggregation of coalescence of the particles.

A suitable working range for solvent:nonsolvent volume ratio is believed to be 1:40-1:1,000,000. An optimal working range for the volume ratios for solvent:nonsolvent is believed to be 1:50-1:200 (volume per volume). Ratios of less than approximately 1:40 resulted in particle coalescence, presumably due to incomplete solvent extraction or else a slower rate of solvent diffusion into the bulk nonsolvent phase.

It will be understood by those of ordinary skill in the art that the ranges given above are not absolute, but instead are interrelated. For example, although it is believed that the solvent:nonsolvent minimum volume ratio is on the order of 1:40, it is possible that microparticles still might be formed at lower ratios such as 1:30 if the polymer concentration is extremely low, the viscosity of the polymer solution is extremely low and the miscibility of the solvent and nonsolvent is high. Thus, the polymer is dissolved in an effective amount of solvent, and the mixture of cytokine, polymer and polymer solvent is introduced into an effective amount of a nonsolvent, so as to produce polymer concentrations, viscosities and solvent:nonsolvent volume ratios that cause the spontaneous and virtually instantaneous formation of microparticles.

Nanospheres and microspheres in the range of 10 nm to 10 μm have been produced using PIN. Using initial polymer concentrations in the range of 1-2% (weight/volume) and solution viscosities of 1-2 centipoise, with a “good” solvent such as methylene chloride and a strong non-solvent such as petroleum ether or hexane, in an optimal 1:100 volume ratio, generates particles with sizes ranging from 100-500 nm. Under similar conditions, initial polymer concentrations of 2-5% (weight/volume) and solution viscosities of 2-3 centipoise typically produce particles with sizes of 500-3,000 nm. Using very low molecular weight polymers (less than 5 kDa), the viscosity of the initial solution may be low enough to enable the use of higher than 10% (weight/volume) initial polymer concentrations which generally result in microspheres with sizes ranging from 1-10 μm. In general, it is likely that concentrations of 15% (weight/volume) and solution viscosities greater than about 3.5 centipoise discreet microspheres will not form but, instead, will irreversibly coalesce into intricate, interconnecting fibrilar networks with micron thickness dimensions.

Although applicants are not bound by any mechanism, it is believed that the surprising therapeutic results obtained in the experiments described herein may arise as a result of one or more of the physiochemical properties of the microparticles. As described above, one of the physiochemical properties of the microparticles used according to the invention is size. The microparticles in some embodiments have a size range from 10 nm to 10 μm. For instance, the microparticles may have an average particle size anywhere in that range, e.g., 10 nm, 100 nm, 1 μm, 5 μm, 10 μm.

Another property of preferred microparticle preparations relates to the bioavailability of the IL-12 released from the microparticle. One challenge associated with generating IL-12 containing microparticles has been to prepare microparticles that release a minimum amount of biologically active IL-12. The microparticles described herein have accomplished that. It has been discovered that microparticles can be prepared wherein the microparticles release between about 0.1% and 20% of the IL-12 in a bioactive form and that this amount of cytokine is sufficient to produce the dramatic biological effects observed in the Examples. In more specific embodiments between about 5% and 10% of the IL-12 released from the microparticle preparation in vivo is bioactive. In yet other embodiments (such as those specifically described in the Examples) about 8% of the IL-12 released from the microparticle preparation in vivo is bioactive.

Yet another property of the microparticles is related to the IL-12 release kinetics. The release of IL-12 over a period of time with a maximum amount being released on day 1 followed by decreasing amounts being released on subsequent days has been shown to provide beneficial effects. The IL-12 released from the microparticles tested in the experiments described in the Examples occurred over a 12 day period, with 100% of the IL-12 being released during that period of time. It is possible, however, to obtain the therapeutic benefits by causing the IL-12 to be released from the microparticles in a variety of time periods, e.g., between about 3 days and 2 months. In some embodiments it is preferred that the IL-12 is released from the microparticle preparation in between about 8 days and 1 month. In other embodiments the IL-12 is released from the microparticle preparation in a between about 12 days and 15 days.

If the IL-12 is being released from the microparticle over a period of time the actual amount of IL-12 released will vary dramatically from one time point to another. For instance, in the experiments described in the Examples, on day one approximately 3400 pg/μg of particle/day is released. By the twelfth day about 60 pg/μg of particle/day is being released. In some embodiments other ranges are observed, e.g., the microparticle preparation may have an IL-12 release rate of between about 250 pg/μg of particle/day and 1000 pg/μg of particle/day. In some embodiments the microparticle preparation has an average IL-12 release rate of about 550 pg/μg of particle/day.

The IL-12 microspheres are delivered directly to the tumor site (in situ). The term “tumor site” as used herein refers to the tumor tissue or the tissue immediately surrounding the tumor, or if the tumor has been surgically removed, the region previously occupied by the tumor. Preferably, the microparticles are injected directly into the tumor site. If the tumor is a tumor of the blood, the microparticles may be delivered to the bloodstream and allowed to circulate. In some cases the cytokine is administered in conjunction (prior to, simultaneously with or following) a medical procedure to remove or kill the tumor cells. The intralesional inoculation of the tumor with cytokine-loaded microspheres prior to the medical procedure allows for maximal stimulation of antitumor immunity without interfering with standard therapy. Accessibility of tumor is not a concern since stereotactic injections could be employed for a large variety of lesions that are not directly accessible.

A “medical procedure to remove or kill the tumor cells” as used herein refers to a surgical procedure, e.g., a surgical resection, treatment with radiation and/or treatment with a cancer medicament, e.g., chemotherapy or immunotherapy.

According to various aspects of the invention, IL-12 may be administered prior to, simultaneously with or after a surgical procedure and/or radiation therapy aimed at treating a cancer. Surgery and radiation are still commonly used to treat a variety of cancers.

As used herein, a “cancer medicament” refers to an agent which is administered to a subject for the purpose of treating a cancer. Cancer medicaments function in a variety of ways. Some cancer medicaments work by targeting physiological mechanisms that are specific to tumor cells. Examples include the targeting of specific genes and their gene products (i.e., proteins primarily) which are mutated in cancers. Such genes include but are not limited to oncogenes (e.g., Ras, Her2, bcl-2), tumor suppressor genes (e.g., EGF, p53, Rb), and cell cycle targets (e.g., CDK4, p21, telomerase). Cancer medicaments can alternately target signal transduction pathways and molecular mechanisms which are altered in cancer cells. Targeting of cancer cells via the epitopes expressed on their cell surface is accomplished through the use of monoclonal antibodies. This latter type of cancer medicament is generally referred to herein as immunotherapy. Still other medicaments, called angiogenesis inhibitors, function by attacking the blood supply of solid tumors. Since the most malignant cancers are able to metastasize (i.e., exist the primary tumor site and seed a distal tissue, thereby forming a secondary tumor), medicaments that impede this metastasis are also useful in the treatment of cancer. Angiogenic mediators include basic FGF, VEGF, angiopoietins, angiostatin, endostatin, TNFα, TNP-470, thrombospondin-1, platelet factor 4, CAI, and certain members of the integrin family of proteins. One category of this type of medicament is a metalloproteinase inhibitor, which inhibits the enzymes used by the cancer cells to exist the primary tumor site and extravasate into another tissue.

Immunotherapeutic agents are medicaments which influence an immune response. These include both passive and active-immunotherapies. One type of passive immunotherapy derives from antibodies or antibody fragments which specifically bind or recognize a cancer antigen. As used herein a cancer antigen is broadly defined as an antigen expressed by a cancer cell. Preferably, the antigen is expressed at the cell surface of the cancer cell. Even more preferably, the antigen is one which is not expressed by normal cells, or at least not expressed to the same level as in cancer cells. Antibody-based immunotherapies may function by binding to the cell surface of a cancer cell and thereby stimulate the endogenous immune system to attack the cancer cell. Another way in which antibody-based therapy functions is as a delivery system for the specific targeting of toxic substances to cancer cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the cancer and non-specific toxicity to normal cells can be minimized. In addition to the use of antibodies which are specific for cancer antigens, antibodies which bind to vasculature, such as those which bind to endothelial cells, are also useful in the invention. This is because generally solid tumors are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

Chemotherapeutic agents as used herein encompass both chemical and biological agents. These agents function to inhibit a cellular activity which the cancer cell is dependent upon for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. Chemotherapeutic agents which can be used according to the invention include but are not limited to Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2'deoxycoformycin), Semustine (methyl-CCNU), Teniposide (VM-26) and Vindesine sulfate.

In some aspects the IL-12 containing microparticles are administered in conjunction with a tumor or cancer antigen. As used herein, the terms “cancer antigen” and “tumor antigen” are used interchangeably to refer to antigens which are differentially expressed by cancer cells and can thereby be exploited in order to target cancer cells. A “cancer antigen” or a “tumor antigen” is a compound, such as a peptide, associated with a tumor or cancer cell surface and which is capable of provoking an immune response when expressed on the surface of an antigen presenting cell in the context of an MHC molecule. Cancer antigens, such as those present in cancer vaccines or those used to prepare cancer immunotherapies, can be prepared from crude cancer cell extracts, as described in Cohen, et al., 1994, Cancer Research, 54:1055, or by partially purifying the antigens, using recombinant technology, or de novo synthesis of known antigens. Cancer antigens can be used in the form of immunogenic portions of a particular antigen or in some instances a whole cell or a tumor mass can be used as the antigen. Such antigens can be isolated (e.g., as defined above) or prepared recombinantly or by any other means known in the art.

The theory of immune surveillance is that a prime function of the immune system is to detect and eliminate neoplastic cells before a tumor forms. A basic principle of this theory is that cancer cells are antigenically different from normal cells and thus elicit immune reactions that are similar to those that cause rejection of immunologically incompatible allografts. Studies have confirmed that tumor cells differ, either qualitatively or quantitatively, in their expression of antigens. For example, “tumor-specific antigens” are antigens that are specifically associated with tumor cells but not normal cells. Examples of tumor specific antigens are viral antigens in tumors induced by DNA or RNA viruses. “Tumor-associated” antigens are present in both tumor cells and normal cells but are present in a different quantity or a different form in tumor cells. Examples of such antigens are oncofetal antigens (e.g., carcinoembryonic antigen), differentiation antigens (e.g., T and Tn antigens), and oncogene products (e.g., HER/neu).

One form of cancer antigen is a whole cell vaccine which is a preparation of cancer cells which have been removed from a subject, treated ex vivo and then reintroduced as whole cells in the subject. Lysates of tumor cells can also be used as cancer vaccines to elicit an immune response. Another form cancer antigen is a peptide vaccine which uses cancer-specific or cancer-associated small proteins to activate T cells. Cancer-associated proteins are proteins which are not exclusively expressed by cancer cells (i.e., other normal cells may still express these antigens). However, the expression of cancer-associated antigens is generally consistently upregulated with cancers of a particular type. Yet another form of cancer antigen is a dendritic cell antigen which includes whole dendritic cells which have been exposed to a cancer antigen or a cancer-associated antigen in vitro. Lysates or membrane fractions of dendritic cells may also be used as cancer antigens. Dendritic cell antigens are able to activate antigen-presenting cells directly.

Cancer antigens include but are not limited to Melan-A/MART-1, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, am11, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmel117}, PRAME, NY-ESO-1, brain glycogen phosphorylase, SSX-1, SSX-2 (HOM-MEL-40), SSX-1, SSX-4, SSX-5, SCP-1 and CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, Smad family of tumor antigens, Imp-1, EBV-encoded nuclear antigen (EBNA)-1, or c-erbB-2.

In some embodiments, cancers or tumors escaping immune recognition and tumor-antigens associated with such tumors (but not exclusively), include acute lymphoblastic leukemia (etv6; aml1; cyclophilin b), B cell lymphoma (Ig-idiotype), glioma (E-cadherin; α-catenin; β-catenin; γ-catenin; p120ctn), bladder cancer (p21ras), billiary cancer (p21ras), breast cancer (MUC family; HER2/neu; c-erbB-2), cervical carcinoma (p53; p21ras), colon carcinoma p21ras; HER2/neu; c-erbB-2; MUC family), colorectal cancer (Colorectal associated antigen (CRC)—C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), hodgkins lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21ras; gp100^{Prel}¹¹⁷) These antigens are also useful according to the invention.

For examples of tumor antigens which are presented by either or both MHC class I and MHC class II molecules, see the following references: Coulie, Stem Cells 13:393-403, 1995; Traversari et al., J. Exp. Med. 176:1453-1457, 1992; Chaux et al., J. Immunol. 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van der Bruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Immunogenetics 43:377-383, 1996; Manici et al., J. Exp. Med. 189:871-876, 1999; Duffour et al., Eur. J. Immunol. 29:3329-3337, 1999; Zorn et al., Eur. J. Immunol. 29:602-607, 1999; Huang et al., J. Immunol.162:6849-6854, 1999; Boël et al., Immunity 2:167-175, 1995; Van den Eynde et al., J. Exp. Med. 182:689-698, 1995; De Backer et al., Cancer Res. 59:3157-3165, 1999; Jäger et al., J. Exp. Med. 187:265-270, 1998; Wang et al., J. Immunol. 161:3596-3606, 1998; Aarnoudse et al., Int. J. Cancer 82:442-448, 1999; Guilloux et al., J. Exp. Med. 183:1173-1183, 1996; Lupetti et al., J. Exp. Med. 188:1005-1016, 1998; Wölfel et al., Eur. J. Immunol. 24:759-764, 1994; Skipper et al., J. Exp. Med. 183:527-534, 1996; Kang et al., J. Immunol. 155:1343-1348, 1995; Morel et al., Int. J. Cancer 83:755-759, 1999; Brichard et al., Eur. J. Immunol 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al., J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et al, J. Immunol. 162:1739-1748, 1999; Kawakami et al., J. Exp. Med. 180:347-352, 1994; Castelli et al., J. Exp. Med. 181:363-368, 1995; Schneider et al., Int. J. Cancer 75:451-458, 1998; Wang et al., J. Exp. Med. 183:1131-1140,1996; Wang et al., J. Exp. Med. 184:2207-2216, 1996; Parkhurst et al., Cancer Research 58:4895-4901, 1998; Tsang et al., J. Natl Cancer Inst 87:982-990, 1995; Correale et al., J Natl Cancer Inst 89:293-300, 1997; Coulie et al., Proc. Natl. Acad. Sci. USA 92:7976-7980, 1995; Wölfel et al., Science 269:1281-1284, 1995; Robbins et al., J. Exp. Med. 183:1185-1192, 1996; Brändle et al., J. Exp. Med. 183:2501-2508, 1996; ten Bosch et al., Blood 88:3522-3527, 1996; Mandruzzato et al., J. Exp. Med. 186:785-793, 1997; Guéguen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999; Fisk et al., J. Exp. Med. 181:2109-2117, 1995; Brossart et al., Cancer Res. 58:732-736, 1998; Röpke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679,1999. These antigens as well as others are disclosed in PCT Application PCT/US98/18601.

Thus, an antigen (one or more) for use in the present invention includes, but is not limited to, proteins or fragments thereof (e.g., proteolytic fragments), peptides (e.g., synthetic peptides, polypeptides), glycoproteins, carbohydrates (e.g., polysaccharides), lipids, glycolipids, hapten conjugates, recombinant DNA, whole organisms (killed or attenuated) or portions thereof, toxins and toxoids (e.g., tetanus, diphtheria, cholera) and/or organic molecules.

As shown in the examples below, the IL-12 microspheres were actually able to cause regression of established tumors in vivo and to prevent metastasis, at dosages which were not toxic. These findings were particularly surprising in view of the prior art. For instance, Cavallo et al, (J. Nat Cancer Instit., 89:1049-1058 (1997)) teaches that when recombinant IL-12 is administered locally to a tumor site, the IL-12 is useful for preventing growth of a newly forming tumor, but has very little effect at all on established tumors. In contrast to this prior art teaching, it has been discovered that when IL-12 is administered directly to the tumor site in the form of a microparticle preparation complete tumor regression was observed in 7 of 10 mice and tumor growth was suppressed in the three remaining mice (Example 3). These surprising results have dramatic therapeutic implications, since many tumors which are most difficult to treat are established tumors. Thus the invention includes methods for effecting tumor regression in a subject having an established tumor. The term “regression” as used herein refers to any reduction in tumor size. This encompasses small reductions in tumor size as well as complete disappearance of detectable tumor cells.

The invention also includes methods for preventing metastasis in a subject. Tumor metastasis involves the spread of tumor cells primarily via the vasculature to remote sites in the body. As used herein “metastases” shall mean tumor cells located at sites discontinuous with the original tumor, usually through lymphatic and/or hematogenous spread of tumor cells. Thus the term metastasis refers to the invasion and migration of tumor cells away from the primary tumor site. A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

The terms “prevent” and “preventing” as used herein with respect to metastasis refer to inhibiting completely or partially the metastasis of a cancer or tumor cell, as well as inhibiting any increase in the metastatic ability of a cancer or tumor cell.

The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers.

The barrier for the tumor cells may be an artificial barrier in vitro or a natural barrier in vivo. In vitro barriers include but are not limited to extracellular matrix coated membranes, such as Matrigel. An in vitro assay for testing the ability of a composition to inhibit tumor cell invasion in a Matrigel invasion assay system is described in detail by Parish, C. R., et al., “A Basement-Membrane Permeability Assay which Correlates with the Metastatic Potential of Tumour Cells,” Int. J. Cancer (1992) 52:378-383. Matrigel is a reconstituted basement membrane containing type IV collagen, laminin, heparan sulfate proteoglycans such as perlecan, which bind to and localize bFGF, vitronectin as well as transforming growth factor-β (TGF-β), urokinase-type plasminogen activator (uPA), tissue plasminogen activator (tPA), and the serpin known as plasminogen activator inhibitor type 1 (PAI-1). Other in vitro and in vivo assays for metastasis have been described in the prior art, see, e.g., U.S. Pat. No. 5,935,850, issued on Aug. 10, 1999, which is incorporated by reference. An in vivo barrier refers to a cellular barrier present in the body of a subject.

Additionally animals that were treated with GM-CSF or IL-2-loaded microspheres experienced a significant albeit less dramatic inhibition or delay in tumor growth. Two of 5 mice that received the GM-CSF microspheres remained tumor-free for six weeks while all mice that were treated with PEG-IL-2-loaded microspheres developed tumors although tumor growth in these mice was delayed compared to the controls. The antitumor effect observed with GM-CSF was surprising. This cytokine induces potent antitumor immunity when used in a prophylactic vaccine setting however it has not been shown to suppress tumor growth directly. Thus in some aspects of the invention methods for suppressing tumor growth by administering to a subject GM-CSF containing microparticles are provided.

In other aspects the invention relates to synergistic combinations of IL-12 and cytokines that augment antigen processing and presentation. It was discovered that when IL-12 is combined with this class of cytokines in the microparticles of the invention that a synergistic reduction in tumor nodules is accomplished. Cytokines that augment antigen processing and presentation include but are not limited to GM-CSF, TNFα and IL-1.

A synergistic amount is that amount which produces an anti-cancer response that is greater than the sum of the individual effects of either the IL-12 or the other cytokine, e.g., GM-CSF alone. For example, a synergistic combination of IL-12 and the GM-CSF provides a biological effect which is greater than the combined biological effect which could have been achieved using each of the components separately.

The IL-12 is delivered in therapeutically effective amounts. An effective mount is that amount which eliminates existing tumors, delays progression of disease, reduces the size of existing tumor, prevents tumor enlargement which would occur without treatment or therapy, delays the onset of tumor formation, delays tumor enlargement, and methods which prevent, reduce or delay metastases. A therapeutically-effective amount can be determined on an individual basis and will be based, at least in part, on consideration of the species of mammal, the mammal's age, sex, size, and health; the time of administration relative to the severity of the disease; and whether a single or multiple controlled-release dose regiments are employed. A therapeutically-effective amount can be determined by one of ordinary skill in the art employing such factors and using no more than routine experimentation.

In some embodiments, the concentration of the IL-12 microparticles is at a dose of about 0.2-70 micrograms for an adult of 70 kg body weight, per day. In other embodiments, the dose is about 3.5-21 micrograms. Preferably, the dosage form is such that it does not substantially deleteriously effect the mammal. The dosage can be determined by one of ordinary skill in the art employing known factors and using no more than routine experimentation.

If the IL-12 microparticles are being administered in combination with cancer antigens or cancer medicaments one of skill in the art can look to any of the many published protocols which describe the administration of these known compounds. For instance, the National Institutes of Health Recombinant DNA Advisory Committee has approved several cancer vaccines using irradiated modified or unmodified tumor cells or other medicaments. For example, see Human Gene Therapy April 1994 Vol. 5, p. 553-563 and references therein to published protocols. These published protocols include: (i) Immunization of Cancer Patients Using Autologous Cancer Cells Modified by Insertion of the Gene for Tumor Necrosis Factor, Principal Investigator S. A. Rosenberg, Human Gene Therapy 3, p. 57-73 (1992); (ii) Immunization of Cancer Patients Using Autologous Cancer Cells Modified by Insertion of the Gene for Interleukin-2, Principal Investigator S. A. Rosenberg, Human Gene Therapy 3, p. 75-90 (1992); (iii) A Pilot Study of Immunization with Interleukin-2 Secreting Allogeneic HLA-A2 Matched Renal Cell Carcinoma Cells in Patients with Advanced Renal Cell Carcinoma, Principal Investigator B. Gansbacher, Human Gene Therapy 3, p. 691-703 (1992); (iv) Immunization with Interleukin-2 Transfected Melanoma Cells. A Phase I-II Study in Patients with Metastatic Melanoma, Human Gene Therapy 4, p. 323-330 (1993); (v) Gene Therapy of Cancer: A Pilot Study of IL-4 Gene Modified Fibroblasts Admixed with Autologous Tumor to Elicit an Immune Response, Principal Investigators M. T. Lotze and I. Rubin, Human Gene Therapy 5, p. 41-55 (1994) (melanoma, renal cell carcinoma, breast, colon); (vi) A protocol was approved Feb. 17, 1995 for colon cancer which combines tumor cells plus fibroblasts engineered to express IL-2 (San Diego Regional Cancer); (vii) Phase I Study of Cytokine-Gene Modified Autologous Neuroblastoma Cells for Treatment of Relapsed/Refractory Neuroblastoma; Principal Investigator: M. K. Brenner; RAC Approval No. 9206-018; (viii) Phase I Study of Non-replicating Autologous Tumor Cell Injections Using Cells Prepared with or without Granulocyte-Macrophage Colony Stimulating Factor Gene Transduction in Patients with Metastatic Renal Cell Carcinoma; Principal Investigator: J. Simons; RAC Approval No. 9303-040; (ix) Phase I Trial of Human Gamma Interferon-Transduced Autologous Tumor Cells in Patients with Disseminated Malignant Melanoma; Principal Investigator: H. F. Seigler; RAC Application No. 9306-043; (x) Phase I Study of Transfected Cancer Cells Expressing the Interleukin-2 Gene Product in Limited Stage Small Cell Lung Cancer; (xi) Immunization of Malignant Melanoma Patients with Interleukin-2 Secreting Melanoma Cells Expressing Defined Allogeneic Histocompatibility Antigens; Principal Investigator: T. K. Das Gupta; RAC Application No. 9309-056. One skilled in the art will recognize that the sections therein regarding patient selection, dose, pretreatment evaluation, concurrent therapy, and treatment of potential toxicity are all applicable here.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants, and optionally other therapeutic ingredients.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The following examples are provided to illustrate specific instances of the practice of the present invention and are not to be construed as limiting the present invention to these examples. As will be apparent to one of ordinary skill in the art, the present invention will find application in a variety of compositions and methods.

### EXAMPLES

In the experiments described herein we evaluated the efficacy of in situ tumor vaccination with IL-12 microspheres in a clinically relevant surgical metastasis model. In this model large primary subcutaneous Line-1 tumors are established and are allowed to spontaneously metastasize to the lungs of the BALB/c mice. The primary subcutaneous tumor is then treated with IL-12 microspheres and is surgically removed one week after treatment. Five weeks after surgery the mice are sacrificed and the lungs are analyzed for the suppression of metastatic nodules. We tested the efficacy of our vaccination strategy using IL-12 alone or IL-12 in combination with GM-CSF. The results of these “neoadjuvant” vaccination studies are set forth below.

Materials and Methods:

Mice and Cell Lines.

Male or female BALB/c mice at 6-8 weeks of age were obtained from Taconic Laboratories (Germantown, N.Y.). CB-17 scid/scid mice were obtained from the Roswell breeding colony. All mice were maintained in microisolation cages (Lab Products, Federalsburg, Mass., USA) under pathogen-free conditions. Animals of both sexes were used in the studies at 8-12 weeks of age. Line-1 (a BALB/c lung alveolar carcinoma cell line) was a gift from Dr. John G. Frelinger (University of Rochester, School of Medicine and Dentistry, Rochester, N.Y.). CB.17 SCID mice were depleted of natural killer (NK) cells by a single i.p. injection of the monoclonal antibody TM-β1 one day prior to the tumor inoculations (a generous gift of Dr. T. Tanaka, Tokyo Metropolitan Institute of Medical Science, Japan) which has been shown to effectively deplete murine NK cells for up to 5 weeks (Tanaka, T., et al., J. Exp. Med. 178:1103-1107, 1993).

Cytokines.

Recombinant human PEG-IL-2 (6×10⁶ IU/mg) was a gift from Chiron, Inc. (Emeryville, Calif.). Recombinant murine IL-12 (2.7×10⁶ units/mg) was donated by Genetics Institute, Inc. (Andover, Mass.) and recombinant murine GM-CSF (7.2×10⁷ units/mg) was donated by Immunex, Inc. (Seattle, Wash.).

Microspheres.

A phase inversion nanoencapsulation technique was used for encapsulation of cytokines as previously described (Mathiowitz, E., et al. Nature 386:410-414, 1997). Briefly, bovine serum albumin (BSA, RIA grade, Sigma Chemical Co., St. Louis, Mo.), polylactic acid (PLA, MW 24,000 and MW 2,000 [1:1, w/w], Birmingham Polymers, Inc, Birmingham, Ala.), and recombinant cytokine in methylene chloride (Fisher, Pittsburgh, Pa.) was rapidly poured into petroleum ether (Fisher, Pittsburgh, Pa.) for formation of microspheres (0.1-10 μm). Microspheres were filtered and lyophilized overnight for complete removal of solvent. Four formulations containing 1% BSA (wt/wt) were produced: 1) control (no cytokines), 2) human PEG-IL-2 (˜10 μg [60,000 IU]/mg PLA), 3) murine IL-12 (˜10 μg [270,000 U]/mg PLA) and 4) murine GM-CSF (˜10 μg [7.2×10⁵ units]/mg PLA). Scanning electron micrographs demonstrated that the microspheres were 1-5 μm in diameter and were easily injectable with a 28.5 gauge needle. The encapsulation efficiencies for the cytokines were extrapolated from the measurements of total protein encapsulated into the microspheres as described (Johnson, O. L., et al. Pharmaceut. Res. 14:730-735, 1997).

Cytokine Release and Bioactivity Assays.

The assay for the quantitation of in vitro cytokine release from the microspheres has been described (Egilmez, N. K., et al. Cancer Immunol. Immunother. 46:21-24, 1998). Briefly, 3 mg of particles in 200 μl of tissue culture medium (Dulbecco's modified Eagle medium+10% fetal calf serum) were incubated in the wells of a 96-well culture plate in triplicate at 37° C. The medium was changed daily for 12-16 consecutive days and the aliquots were stored at 4° C. The quantity of cytokine in the medium was either determined by ELISA (R & D Systems, Minneapolis, Minn.), or in the case of PEG-IL-2, by a bioactivity assay using an IL-2-dependent murine T cell line proliferation assay (Egilmez, N. K., et al. Cancer Immunol. Immunother. 46:21-24, 1998). The bioactivity assay for recombinant murine IL-12 was performed using a murine splenocyte proliferation assay as described (Mattner, F., et al. Eur. J. Immunol. 23:2202-2208, 1993).

### Example 1

### Cytokines are Efficiently Encapsulated into and Released from the PLA Microspheres.

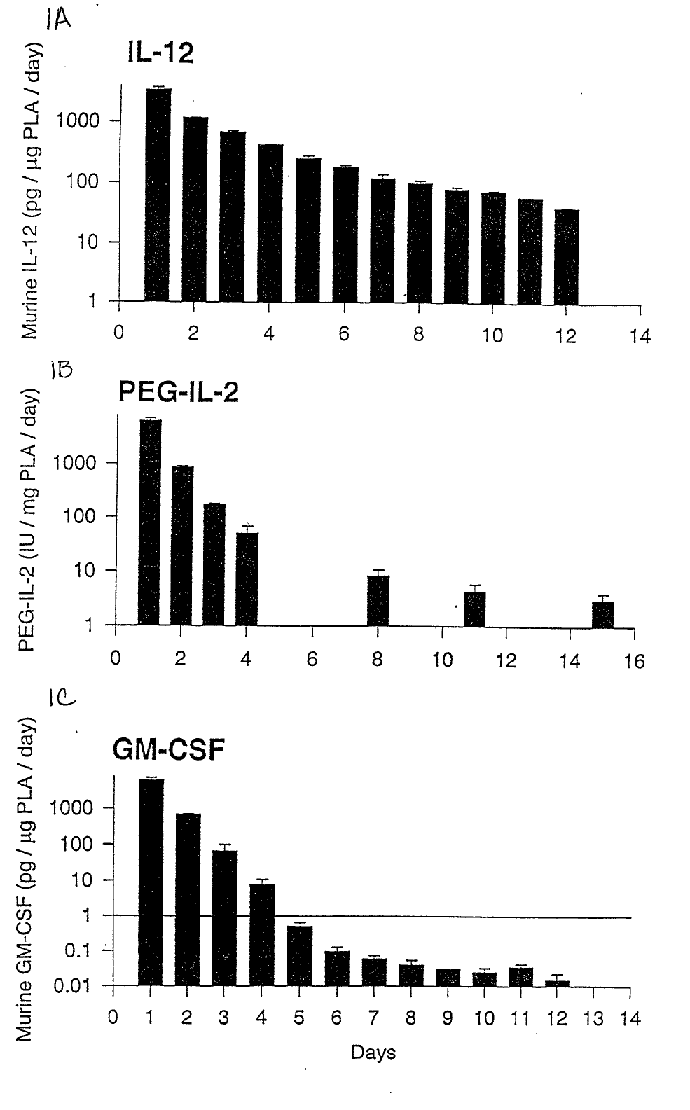

The encapsulation efficiencies and in vitro release patterns of three different recombinant cytokines were evaluated. Encapsulation efficiency into PLA microspheres was determined to be 67±1% for murine heterodimeric IL-12 (MW 70 kD), 95±6% for murine GM-CSF (MW 23 kD) and 65±6% for human PEG-IL-2 (MW 15-94 kD). The in vitro release patterns of IL-12, PEG-IL-2 and GM-CSF from the microspheres are shown in FIGS. 1A, 1b, and 1C respectively. The initial release of cytokines is followed by a rapid decline with an eventual stabilization of the release kinetics after day 7. Both PEG-IL-2 and IL-12 that were released from the cytokines were shown to be bioactive in vitro (FIG. 1). The results indicate that significant quantities of cytokine can be released from the microspheres for at least 12 days, but that the absolute quantities and the release rates vary depending on the particular cytokine that is encapsulated.

### Example 2

### Co-injection of Cytokine-loaded Microspheres with a Single-cell Suspension of Live Tumor Cells Suppresses Tumor Engraftment.

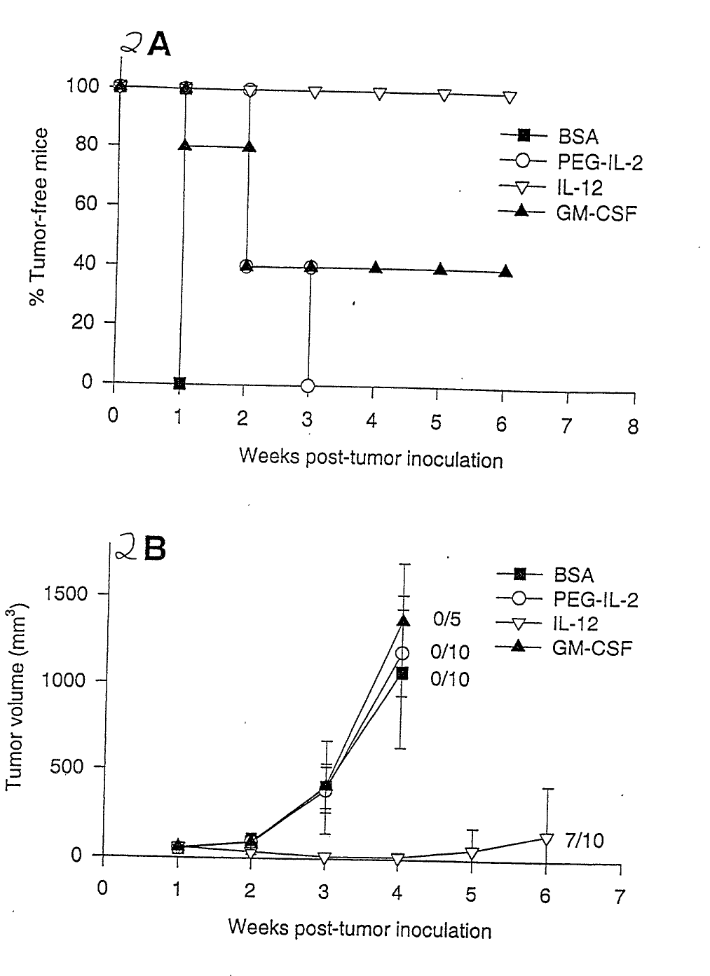

The in vivo immunotherapeutic potential of the cytokine-loaded microspheres was initially tested by co-injecting the microspheres with live Line-1 tumor cells subcutaneously into BALB/c mice. Line-1 is a lung alveolar cell carcinoma that arose spontaneously in a female BALB/c mouse (Yuhas, J. M. and Pazmiño, N. H. Cancer Res. 34:2005-2010, 1974). This poorly immunogenic tumor grows rapidly and progressively in the subcutaneous site and ultimately metastasizes to the lungs of the inoculated mice (Yuhas, J. M. and Pazmiño, N. H. Cancer Res. 34:2005-2010, 1974). Mice were injected with Line-1 cells mixed with either control (BSA) or cytokine-loaded microspheres and tumor growth was monitored weekly. The results are shown in FIG. 2A. At the tumor cell dose used, all mice in the control group (BSA microspheres) developed palpable tumors by day 3 with tumors reaching a diameter of 5 mm within 7-8 days. In contrast, all mice that were treated with the IL-12-loaded microspheres remained tumor-free for at least 6 weeks. Mice that were treated with GM-CSF or PEG-IL-2-loaded microspheres experienced a significant albeit less dramatic inhibition or delay in tumor growth. Two of 5 mice that received the GM-CSF microspheres remained tumor-free for six weeks while all mice that were treated with PEG-IL-2-loaded microspheres developed tumors although tumor growth in these mice was delayed compared to the controls. The antitumor effect observed with GM-CSF was surprising. This cytokine induces potent antitumor immunity when used in a prophylactic vaccine setting however it has not been shown to suppress tumor growth directly (Dranoff, G. J. Clin. Oncol. 16:2548-2556, 1998). Interestingly, IL-2 which has been shown to induce tumor suppression in numerous murine tumor models had only a weak antitumor effect here. The observed effects (or lack thereof) could be related to the dose and the release pattern of the particular cytokine delivered by the microspheres. Regardless of the relative antitumor efficacy of individual cytokines, the above results establish that the cytokines released from the microspheres are biologically active in vivo, and that tumor growth can be completely arrested when IL-12-loaded microspheres are injected at the same time that tumors are inoculated into mice.

### Example 3

### IL-12 but Not PEG-IL-2 or GM-CSF-loaded Microspheres Induce Complete Regression of Established and Progressively Growing Tumors Following a Single Intratumoral Injection.

The ability to prevent tumor engraftment is a useful initial screen for evaluating the potential of an anticancer therapy. However, a more clinically relevant approach involves treating established tumors to determine whether or not the local and sustained release of cytokines from the microspheres is able to induce tumor remission and not simply prevent its engraftment. To this end, mice were inoculated with Line-1 cells subcutaneously and the tumors were allowed to grow to ˜4 mm in diameter prior to treatment. These tumors were then injected with cytokine-loaded microspheres and tumor growth was monitored weekly. In these experiments the dose of microspheres was increased significantly as compared to that used in the co-engraftment studies (2 mg as opposed to 50 μg per injection) since the number of tumor cells within the established tumors is greater and established tumors are more difficult to suppress and eradicate. The results are shown in FIG. 2B. There was no significant difference between the growth patterns of tumors treated with control (BSA-loaded) microspheres and PEG-IL-2 or GM-CSF-loaded microspheres where tumors grew progressively. However, a single intratumoral injection of IL-12-loaded microspheres promoted complete tumor regression in 7 of 10 mice and tumor growth was suppressed in the three remaining mice. These results demonstrate that the sustained release of IL-12 from the microspheres can induce potent antitumor activity in a clinically relevant setting.

### Example 4

### Tumor Regression is Accompanied with the Development of Protective Antitumor Immunity, the Potency of which is Dependent on the Method of Vaccination.

The ultimate goal of immunotherapy is to promote the development of long-term systemic antitumor immunity to prevent recurrence of tumors which can not be achieved with conventional treatments such as chemotherapy and radiation. To test whether IL-12 delivered by microspheres directly into existing tumors is able to promote protective antitumor immunity, mice that were able to reject established subcutaneous tumors following treatment with IL-12-loaded microspheres were challenged with live tumor cells at a different site 5-6 weeks after the original tumor had completely regressed. The results of this experiment are shown in Table 1. Of the 15 vaccinated mice that were challenged, 12 rejected the tumor (80%) suggesting the development of potent protective antitumor immunity in these mice.

TABLE 1
The potency of the protective antitumor immunity induced by the
IL-12-loaded microspheres is dependent on the vaccination method.

| | % Tumor rejection after |
|---|---|
| Method of vaccination | challenge^{d} |
| ^{a}Established tumor + IL-12 microspheres | 80%(12/15) |
| ^{b}Live Line-1 cells + IL-12 microspheres | 57% (8/14) |
| ^{c}Irradiated Line-1 cells + IL-12 microspheres | 10% (1/10) |
| Irradiated Line-1 cells alone | 10% (1/10) |
| No treatment | 0% (0/5) |

^{a}Tumor was injected with 2 mg of microspheres
^{b}1 × 10⁶ tumor cells + 50 μg of microspheres
^{c}2 × 10⁶ tumor cells + 50 μg microspheres
^{d}Mice challenged with 1 × 10⁴ tumor cells injected subcutaneously

In parallel experiments, the antitumor efficacy of different vaccination strategies with mixtures of IL-12-loaded microspheres and single-cell suspensions of tumor cells (live or irradiated) were compared to direct intratumoral (in situ) treatments of progressively growing tumors. As shown in Table 1, vaccination of mice with mixtures of IL-12 microspheres and live Line-1 cells provided less protection from a subsequent tumor challenge than in situ vaccination (57 vs 80%). Only 10% of the mice were protected from tumor challenge with an irradiated cell/IL-12 microsphere vaccine which was identical to that obtained with irradiated cells alone. In the control non-vaccinated group none of the mice were able to reject tumor challenge.

To determine if the immunity provoked by the cytokine-loaded microspheres was tumor-specific, mice that rejected subcutaneous Line-1 tumors following vaccination in situ were challenged either with Line-1 or Colon 26 (an unrelated colon tumor cell line derived from BALB/c mice) cells and tumor growth was monitored. While 6 of 6 mice vaccinated with Line-1 rejected the Line-1 challenge, only 1 of 6 vaccinated mice rejected a challenge with Colon 26 tumor cells (Table 2). Non-vaccinated control mice did not reject challenges with either tumor cell line. These results demonstrate that the systemic antitumor immunity induced by the IL-12-loaded microspheres was tumor-specific.

TABLE 2
The antitumor immunity that results from vaccination with IL-12
microspheres is tumor-specific.

| Method of | | |
|---|---|---|
| vaccination | Tumor challenge | % Tumor rejection |
| ^{a}Established Line-1 tumors + | Line-1 | 100 (6/6) |
| IL-12 microspheres | | |
| ^{a}Established Line-1 tumors + | Colon 26 | 17 (1/6) |
| IL-12 microspheres | | |
| No treatment | Line-1 | 0 (0/5) |
| No treatment | Colon 26 | 0 (0/5) |

^{a}treated with 2 mg of microspheres, challenged with 1 × 10⁴ live cells.

### Example 5

### IL-12-loaded Microspheres Stimulate an NK Cell-dependent Delay in Tumor Growth but Fail to Induce Complete Tumor Regression in CB.17 SCID Mice.

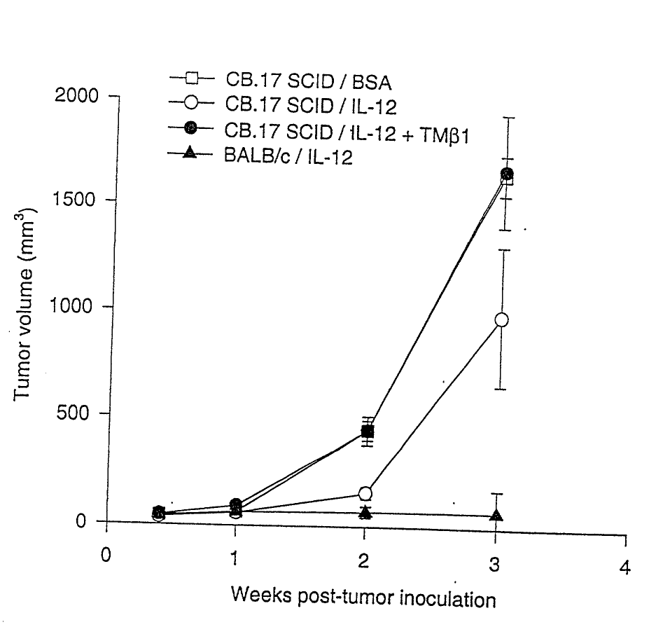

To determine whether the microsphere-mediated tumor regression observed here was induced by T-lymphocytes and NK cells through an IFNγ-dependent mechanism, microsphere vaccination experiments were repeated in CB.17 SCID mice which lack functional B and T-lymphocytes. Mice with established subcutaneous tumors were treated with intratumoral injections of IL-12-loaded microspheres and tumor growth was monitored. The results are shown in FIG. 3. Treatment with IL-12-loaded microspheres delayed tumor growth by 1 week in the CB.17 SCID mice but failed to promote tumor regression. The limited antitumor response observed in the CB.17 SCID mice was shown to be NK-cell dependent since the depletion of the mouse NK cells with the monoclonal antibody TMβ1 resulted in the loss of the tumor suppressive activity. In contrast, a significant tumor suppression was observed in the immunocompetent BALB/c mice with tumors regressing completely in 3 of 5 mice.

### Example 6

### Intratumoral Administration of Microspheres is Critical to Tumor Eradication and Treatment with IL-12-loaded Microspheres is Superior to Bolus Injections of Free IL-12.

To determine whether local release of IL-12 from the microspheres to the tumor microenvironment was necessary, mice were inoculated with IL-12-loaded microspheres either intratumorally or on the contralateral side of tumor-bearing mice and tumor growth was monitored. The results are shown in Table 3. In this experiment 53% of the tumors regressed completely following intratumoral delivery whereas none of the tumors regressed when the microspheres were injected on the contralateral flank of tumor-bearing mice. Moreover, a single intratumoral injection of free IL-12 at a dose equal to that delivered by the microspheres resulted in the regression of tumors in only 20% of the animals while i.p. delivery of free IL-12 did not promote any tumor regression. These results demonstrate that local and sustained delivery of IL-12 to tumors is superior to local or systemic bolus delivery.

TABLE 3
Local and sustained delivery of IL-12 is critical to cure of
established tumors in the Line-1/BALB/c model.

| Method of delivery | Location | % Tumor cure^{c} |
|---|---|---|
| ^{a}Microspheres | Intratumoral | 53 (8/15) |
| ^{a}Microspheres | Contralateral | 0 (0/5) |
| ^{b}Free cytokine | Intratumoral | 20 (1/5) |
| ^{b}Free cytokine | Intraperitoneal | 0 (0/5) |

^{a}2 mg of microspheres (˜29,000 units of IL-12)
^{b}1 μg of free IL-12 (˜27,000 units of IL-12)
^{c}defined as complete regression with no evidence of recurrence for at least 6 weeks

### Example 7

### Treatment of Established Subcutaneous Tumors with IL-12-loaded Microspheres Suppresses both the Growth of Subcutaneous Tumors and the Distant Metastatic Lesions.

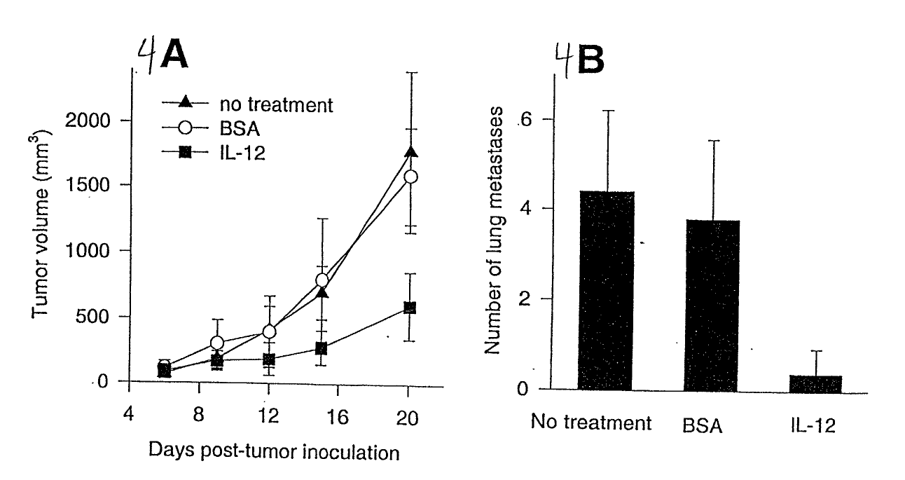

Line-1 cells, when injected subcutaneously, metastasize spontaneously to the lungs of the BALB/c mice (Yuhas, J. M. and Pazmiño, N. H. Cancer Res. 34:2005-2010, 1974). To determine whether treatment of established subcutaneous tumors with IL-12-loaded microspheres could also promote the suppression of metastasis, mice with established large (˜7-8 mm) subcutaneous tumors were treated with IL-12-loaded microspheres and their lungs were analyzed 2 weeks after treatment. The results are shown in FIG. 4. Treatment with IL-12-loaded microspheres induced significant suppression of tumor growth compared to treatment with BSA-loaded microspheres (FIG. 4A). Although the primary subcutaneous tumors were suppressed, treatment here did not result in complete regression due to the larger tumor innoculum and the greater size of the tumors at the time of treatment compared to previous experiments. More interestingly however, the examination of the lungs two weeks after treatment revealed significant suppression of lung metastasis in the IL-12 treated animals as compared to the controls (FIG. 4B). These results demonstrate that the local treatment of primary tumors with IL-12-loaded microspheres can suppress both the growth of the primary tumor and metastasis to distant sites. Whether the anti-metastatic effect observed here was due to the systemic presence of the cytokine released by the microspheres or to the development of systemic antitumor immunity that resulted from a release of the cytokine into the tumor microenvironment was not determined. The results shown in Tables 1 and 2 establish that intratumoral delivery of IL-12 microspheres induces the development of a potent tumor-specific systemic immunity. Moreover, the results summarized in Table 3 demonstrate that when the IL-12 microspheres are injected contralateral to tumors, tumor regression is not induced. Together, these data support the notion that the suppression of lung metastasis observed here is most likely mediated by the development of a systemic antitumor immunity and is not simply due to systemic release of IL-12 from the microspheres.

### Example 8

### In situ Tumor Vaccination with IL-12 Microspheres in Another Clinically Relevant Surgical Metastasis Model.

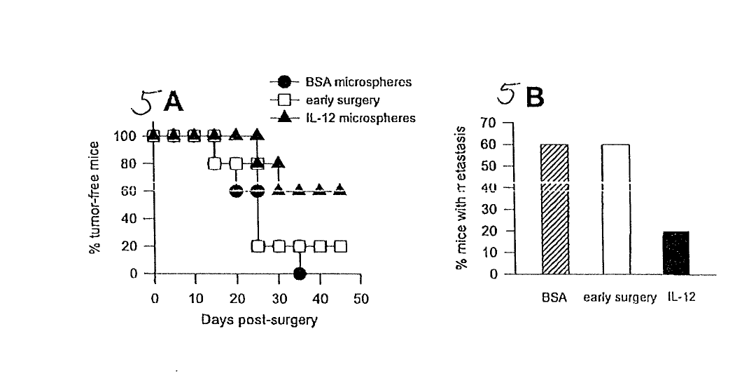

Preoperative neoadjuvant vaccination with IL-12 microspheres prevents recurrence at the surgical site and reduces lung metastasis. Subcutaneous tumors were allowed to reach a size of ˜100 mm³ at which time intratumoral treatment with microspheres (2 mg/tumor) was administered. The tumors were then surgically resected one week after vaccination and recurrence at the subcutaneous site and the development of lung metastasis was monitored. The results are shown in FIG. 5. Tumors recurred at the primary site in only 40% of the mice that were vaccinated with IL-12 microspheres. In control groups where mice were either vaccinated with BSA microspheres or the surgical resection of tumors was performed (without vaccination) at the time the other groups were vaccinated (early surgery) tumors recurred in 100% or 80% of the cases, respectively. Mice were sacrificed 6-7 weeks after surgery (or earlier when recurrence was observed) and lungs were inspected for tumor nodules. Lung metastasis was observed in only 20% of the mice that were vaccinated with IL-12 microspheres. In the control groups 60% of the mice had visible evidence of macroscopic disease. These results establish that vaccination at the primary site results in the development of potent systemic antitumor immunity that suppressed the growth of distant lung nodules effectively.

### Example 9

### In vivo Synergistic Results Obtained with IL-12 and GM-CSF Microspheres.

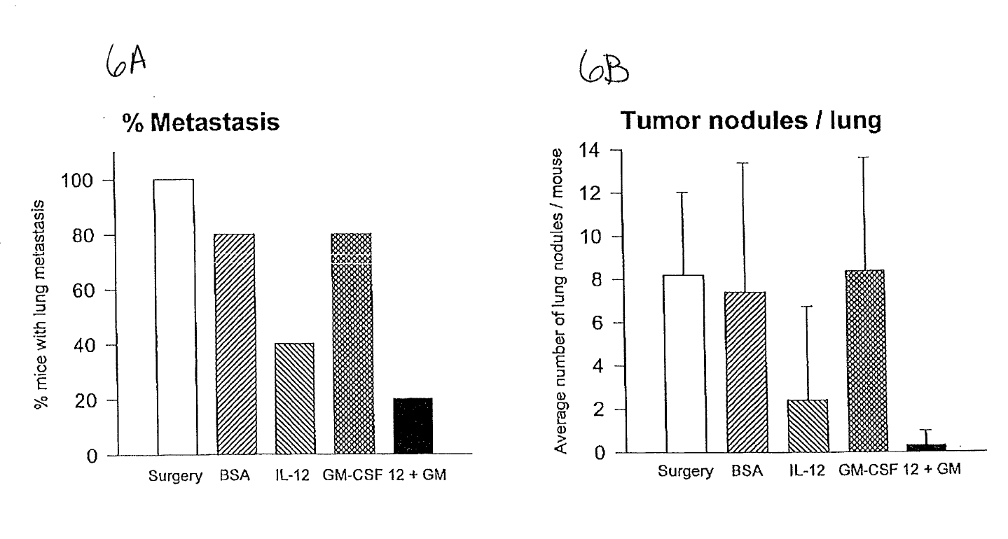

Preoperative neoadjuvant vaccination with IL-12+GM-CSF microspheres is superior to vaccination with either cytokine alone. The combination of cytokines produces a synergistic effect on the inhibition of tumor nodule development. We tested the efficacy of combined vaccination with IL-12 and GM-CSF-loaded microspheres to see whether the antitumor efficacy of our approach could be improved in the surgical metastasis model. The results are shown in FIG. 6. In the control groups (early surgery and BSA microspheres) 80-100% of the mice developed lung metastasis. Treatment with GM-CSF alone was not effective with 80% of the mice developing lung metastasis. IL-12 microspheres were again effective with only 40% of the mice positive for lung tumors. On the other hand, combination therapy with IL-12 and GM-CSF resulted in the most potent suppression of lung metastasis with only 20% of the mice developing lung lesions. In this experiment, the number of nodules/lung were also noted. The data are presented in Table 4.

TABLE 4

| | control | | | |
|---|---|---|---|---|
| Surgery alone | microspheres | IL-12 alone | GM alone | IL-12 + GM |
| 8.2 | 7.4 | 2.4 | 8.4 | 0.3 |

These data underline the potency of the combination treatment where only one mouse out of five had a single lung nodule in the IL-12+GM-CSF group. In these experiments recurrence was minimal, restricted to one or two mice in the control and GM-CSF alone groups due to improved surgical technique.

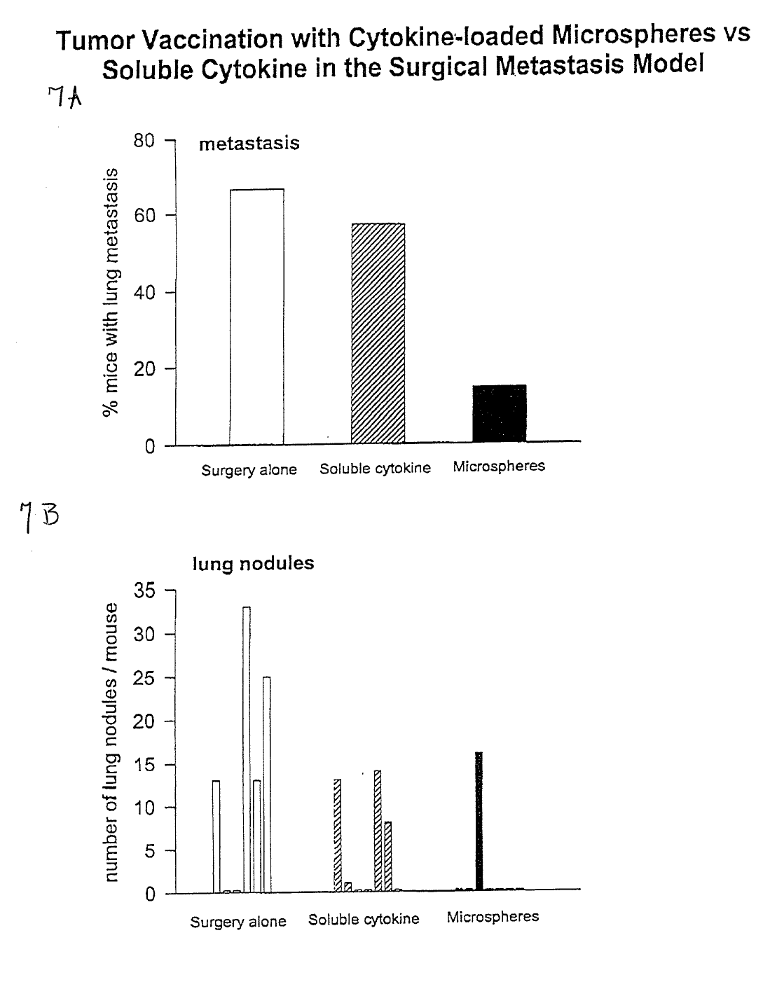

Sustained release of IL-12+GM-CSF from the microspheres is superior to bolus delivery of soluble cytokine in the surgical metastasis model. The sustained presence of cytokines in the tumor environment is critical to the development of a proper immune response. Since most cytokines have short in vivo half-lives, sustained release from polymer microspheres represents an advantage over bolus injections of soluble cytokine. Although repeated injections of soluble cytokine is possible in the case of tumors that are close to the surface of the skin, repeated injections are not clinically feasible in the case of internal tumors such as colon, liver, lung, brain etc. Polymer microspheres also have the advantage that physiologically relevant amounts of cytokine can be delivered locally to the tumor vaccination site without inducing systemic toxicity or generalized immunosuppression as seen with bolus i.v. delivery of the cytokine. We compared the ability of IL-12+GM-CSF microspheres to that of bolus soluble cytokine delivered intratumorally to induce antitumor immunity in the surgical metastasis model. The results are shown in FIG. 7. The mice were vaccinated with either a) no treatment (early surgery), b) IL-12+GM-CSF microspheres or c) by a bolus injection of soluble IL-12+GM-CSF (a dose equal to that delivered by the microspheres). Metastasis to the lungs was evaluated 5 weeks after surgical removal of the primary tumor as above. The results shown below in FIG. 7 establish that microsphere-based delivery is superior to soluble cytokine in the surgical metastasis model.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for in situ tumor vaccination of a subject, comprising
administering to a tumor of a subject an effective amount for preventing tumor growth of a microparticle preparation containing IL-12, wherein an antigen is not co-administered to the subject.

2. The method of claim 1, wherein the microparticle preparation is administered to the subject prior to a medical procedure to remove or kill the tumor cells.

3. The method of claim 1, wherein the microparticle preparation is administered to the subject during or following a medical procedure to remove or kill the tumor cells.

4. The method of claim 3, wherein the medical procedure is a surgical procedure.

5. The method of claim 3, wherein the medical procedure is a chemotherapeutic procedure.

6. The method of claim 3, wherein the medical procedure is an immunotherapeutic procedure.

7. A method for in situ tumor vaccination of a subject, comprising:
administering to a site of a tumor of a subject an effective amount for preventing tumor growth of a microparticle preparation containing IL-12, the microparticles of the microparticle preparation have an average particle size of between 10 nanometers and 10 microns.

8. The method of claim 7, further comprising administering to the subject a tumor antigen.

9. The method of claim 8, wherein the tumor antigen is a tumor cell suspension.

10. The method of claim 8, wherein the tumor antigen is a purified antigen.

11. The method of claim 8, wherein the tumor antigen is a recombinant antigen.

12. The method of claim 7, wherein between about 0.1% and 20% of the IL-12 released from the microparticle preparation in vivo is bioactive.

13. The method of claim 7, wherein between about 5% and 10% of the IL-12 released from the microparticle preparation in vivo is bioactive.

14. The method of claim 7, wherein about 8% of the IL-12 released from the microparticle preparation in vivo is bioactive.

15. The method of claim 7, wherein the microparticle preparation has an IL-12 release rate of between about 60 pg/μg of particle/day and 3400 pg/μg of particle/day.

16. The method of claim 7, wherein the microparticle preparation has an IL-12 release rate of between about 250 pg/μg of particle/day and 1000 pg/μg of particle/day.

17. The method of claim 7, wherein the microparticle preparation has an average IL-12 release rate of about 550 pg/μg of particle/day.

18. The method of claim 7, wherein IL-12 is released from the microparticle preparation over a period of between about 3 days and 2 months.

19. The method of claim 7, wherein IL-12 is released from the microparticle preparation over a period of between about 8 days and 1 month.

20. The method of claim 7, wherein IL-12 is released from the microparticle preparation over a period of between about 12 days and 15 days.

21. A method for in situ tumor vaccination of a subject, comprising:
administering to a site of a tumor of a subject an effective amount for preventing tumor growth of a microparticle preparation containing IL-12, the microparticle of the microparticle preparation having been prepared by phase inversion nanoencapsulation.

22. The method of claim 21, further comprising administering to the subject a tumor antigen.

23. The method of claim 22, wherein the tumor antigen is a tumor cell suspension.

24. The method of claim 22, wherein the tumor antigen is a purified antigen.

25. The method of claim 22, wherein the tumor antigen is a recombinant antigen.

26. The method of claim 21, wherein between about 0.1% and 20% of the IL-12 released from the microparticle preparation in vivo is bioactive.

27. The method of claim 21, wherein between about 5% and 10% of the IL-12 released from the microparticle preparation in vivo is bioactive.

28. The method of claim 21, wherein the microparticle preparation has an IL-12 release rate of between about 60 pg/μg of particle/day and 3400 pg/μg of particle/day.

29. The method of claim 21, wherein the microparticle preparation has an IL-12 release rate of between about 250 pg/μg of particle/day and 1000 pg/μg of particle/day.

30. The method of claim 21, wherein IL-12 is released from the microparticle preparation over a period of between about 3 days and 2 months.

31. The method of claim 21, wherein IL-12 is released from the microparticle preparation over a period of between about 12 days and 15 days.

32. A method for in situ tumor vaccination of a subject, comprising:
administering to a site of a tumor of a subject an effective amount for preventing tumor growth of a microparticle preparation containing IL-12, wherein the microparticle preparation is administered to the subject during or following a medical procedure to remove or kill the tumor cells.

33. The method of claim 32, wherein the medical procedure is a surgical procedure.

34. The method of claim 32, wherein the medical procedure is a chemotherapeutic procedure.

35. The method of claim 32, wherein the medical procedure is an immunotherapeutic procedure.

36. The method of claim 32, further comprising administering to the subject a tumor antigen.

37. The method of claim 36, wherein the tumor antigen is a tumor cell suspension.

38. The method of claim 36, wherein the tumor antigen is a purified antigen.

39. The method of claim 36, wherein the tumor antigen is a recombinant antigen.

40. The method of claim 32, wherein the microparticles of the microparticle preparation have an average particle size of between 10 nanometers and 10 microns.

41. The method of claim 32, the microparticle of the microparticle preparation having been prepared by phase inversion nanoencapsulation.

42. The method of claim 32, wherein between about 0.1% and 20% of the IL-12 released from the microparticle preparation in vivo is bioactive.

43. The method of claim 32, wherein between about 5% and 10% of the IL-12 released from the microparticle preparation in vivo is bioactive.

44. The method of claim 32, wherein the microparticle preparation has an IL-12 release rate of between about 60 pg/μg of particle/day and 3400 pg/μg of particle/day.

45. The method of claim 32, wherein the microparticle preparation has an IL-12 release rate of between about 250 pg/μg of particle/day and 1000 pg/μg of particle/day.

46. The method of claim 32, wherein IL-12 is released from the microparticle preparation over a period of between about 3 days and 2 months.

47. The method of claim 32, wherein IL-12 is released from the microparticle preparation over a period of between about 12 days and 15 days.

48. A method for preventing tumor metastasis in a subject, comprising:
administering to a site of a tumor of a subject in need thereof an effective amount for preventing tumor metastasis of a microparticle preparation containing IL-12.

49. The method of claim 48, further comprising administering to the subject a tumor antigen.

50. The method of claim 49, wherein the tumor antigen is a tumor cell suspension.

51. The method of claim 49, wherein the tumor antigen is a purified antigen.

52. The method of claim 49, wherein the tumor antigen is a recombinant antigen.

53. The method of claim 48, wherein the microparticles of the microparticle preparation have an average particle size of between 10 nanometers and 10 microns.

54. The method of claim 48, the microparticle of the microparticle preparation having been prepared by phase inversion nanoencapsulation.

55. The method of claim 48, wherein between about 0.1% and 20% of the IL-12 released from the microparticle preparation in vivo is bioactive.

56. The method of claim 48, wherein between about 5% and 10% of the IL-12 released from the microparticle preparation in vivo is bioactive.

57. The method of claim 48, wherein the microparticle preparation has an IL-12 release rate of between about 60 pg/μg of particle/day and 3400 pg/μg of particle/day.

58. The method of claim 48, wherein the microparticle preparation has an IL-12 release rate of between about 250 pg/μg of particle/day and 1000 pg/μg of particle/day.

59. The method of claim 48, wherein IL-12 is released from the microparticle preparation over a period of between about 3 days and 2 months.

60. The method of claim 48, wherein IL-12 is released from the microparticle preparation over a period of between about 12 days and 15 days.

61. A method for effecting tumor regression in a subject, comprising:
administering to a site of a tumor of a subject in need thereof an effective amount for effecting tumor regression of a microparticle preparation containing IL-12.

62. A method for in situ tumor vaccination of a subject, comprising
administering to a tumor of a subject a microparticle preparation containing an effective amount of IL-12 and a cytokine that augments antigen processing and presentation, wherein the effective amount of IL-12 and the cytokine that augments antigen processing and presentation results in a synergistic prevention of tumor cell growth.

63. The method of claim 62, wherein the IL-12 and the cytokine that augments antigen processing and presentation results in a synergistic prevention of metastasis.

64. The method of claim 62, wherein the cytokine that augments antigen processing and presentation is GM-CSF.

65. A method for in situ tumor vaccination of a subject, comprising:
administering to a site of a tumor of a subject an effective amount for preventing tumor growth of a microparticle preparation containing IL-12, wherein between about 0.1% and 20% of the IL-12 released from the microparticle preparation in vivo is bioactive.

66. The method of claim 65, wherein between about 5% and 10% of the IL-12 released from the microparticle preparation in vivo is bioactive.

67. The method of claim 65, wherein about 8% of the IL-12 released from the microparticle preparation in vivo is bioactive.

68. A method for in situ tumor vaccination of a subject, comprising:
administering to a site of a tumor of a subject an effective amount for preventing tumor growth of a microparticle preparation containing IL-12, wherein the microparticle preparation has an IL-12 release rate of between about 60 pg/μg of particle/day and 3400 pg/μg of particle/day.

69. The method of claim 68, wherein the microparticle preparation has an IL-12 release rate of between about 250 pg/μg of particle/day and 1000 pg/μg of particle/day.

70. The method of claim 68, wherein the microparticle preparation has an IL-12 release rate of about 550 pg/μg of particle/day.

71. The method of claim 68, wherein IL-12 is released from the microparticle preparation over a period of between about 3 days and 2 months.

72. The method of claim 68, wherein 1L-12 is released from the microparticle preparation over a period of between about 8 days and 1 month.

73. The method of claim 68, wherein IL-12 is released from the microparticle preparation over a period of between about 12 days and 15 days.
